(19) United States
(12) Patent Application Publication
(10) Pub. No.: US 2003/0055494 A1
(43) Pub. Date: Mar. 20, 2003

(54) Adventitial fabric reinforced porous prosthetic graft

(76) Inventors: Deon Bezuidenhout, Vredehoek (ZA); Ross Millam, Rondebosch (ZA); Mark Yeoman, Rondebosch (ZA); Peter Zilla, Camps Bay (ZA)

Correspondence Address: Kenneth J. Collier, Medtronic, Inc., 710 Medtronic Parkway N.E., Minneapolis, MN, 55432, US

(21) Appl. No.: 10/201,498

(22) Filed: Jul. 23, 2002

Related U.S. Application Data
(60) Provisional application No. 60/308,471, filed on Jul. 27, 2001.

(51) Int. Cl. A61F 2/06

(52) U.S. Cl. 623/1.39; 623/1.44; 623/1.5; 623/1.53; 700/130

(57) ABSTRACT

A vascular prosthesis is constructed of an inner porous tube which allows uninterrupted cellular growth and which is connected to an adventitial sock surrounding the porous tube. The adventitial sock produces a non-linear elastic response to stress-strain on the prosthesis to optimize compliance and prevent over dilatation.

### FIELD OF THE INVENTION

This invention is directed to a vascular prosthesis having an inner layer with a well defined core structure to allow uninterrupted ingrowth of connective tissue into the wall of the prosthesis and an outer reinforcing layer having non-linear mechanical properties which when combined with the porous substructure has mechanical properties which resemble that of the host vessel.

### BACKGROUND OF THE INVENTION

Since the early 1950's, when one observer noted an essentially thrombus-free silk thread in a prosthesis explanted from a dog, various polymeric materials have been evaluated for use for porous vascular prostheses. Most commercially available synthetic vascular grafts presently in use are made from either expanded polytetrafluorethylene (e-PTFE), or woven, knitted, or velour design polyethylene terephthalate (PET) or Dacron. These conventional prosthetic vascular grafts do not permit unrestricted vessel ingrowth from surrounding tissue due mostly to ingrowth spaces that are too narrow or discontinuous. When used for smaller diameters, these grafts often fail early due to occlusion by thrombosis or kinking, or at a later stage because of an anastomotic or neointimal hyperplasia (exuberant muscle growth at the interface between artery and graft. Compliance mismatch between the host artery and the synthetic vascular prosthesis, which may result in anastomotic rupture, disturbed flow patterns and increased stresses is thought to be a causative factor in graft failure. Other causative factors may include the thrombogenecity of the grafts or the hydraulic roughness of the surface, especially in crimped grafts.

There has thus been a need for development of a long-term patent graft, which allows for cellular ingrowth as well as displaying the right mechanical characteristics. For example, a structure, which allows or promotes cellular ingrowth, is one important characteristic for the graft. This cellular ingrowth promotes the growth of a confluent endothelial layer on the inner surface of the graft which helps prevent thrombotic surface effects and reduce shear and turbulent forces in the blood flow through the graft. Although some research has focused on wound external reinforcing, it was found that such strengthening of a graft caused compression through the graft wall and high stress concentrations in the region of the wound reinforcing. Stiffening and reduced compliance was a further result. What was needed was a highly porous prosthetic vascular graft, which would allow for unrestricted vessel ingrowth from surrounding tissue, as well as a graft, which matched the mechanical requirements and dynamic compliance, to that of the host.

### SUMMARY OF THE INVENTION

The invention is directed to a vascular graft prosthesis comprising a bi-layer concept and structure to minimize mechanical and compliance mismatch in host vessels. The bi-layer has an inner porous tube or similar structure, which allows uninterrupted cellular growth connected to an adventitial outer layer, which provides a non-linear elastic response and uninterrupted in-growth space into the porous sub-structure. Several different methods can be used to produce the structure and function of the invention. In one method, the structure comprises a super porous polyurethane substructure and an adventitial fabric-reinforcing sock. The sock may be manufactured using different techniques and materials.

Another embodiment includes a vascular graft prosthesis having a bi-layer wall structure configured to optimize mechanical compliance in a host vessel. The structure includes an inner material shaped as a tube structure, which allows uninterrupted cellular growth, and an outer adventitial material. The outer adventitial material is connected to the inner tube, and the adventitial material is characterized by a non-linear elastic response to strain.

Another aspect of the invention is a method of using geometrical properties of a textile fabric structure to produce a non-linear elastic response in a porous multi-layer vascular graft. The method comprises the steps of configuring an outer textile fabric layer into a tubular form, and then arranging the textile fabric layer around a porous inner layer. In this manner, the method permits cellular ingrowth to be promoted while also minimizing compliance mismatch.

Finite element methods and optimization tools are used to determine the specific requirements of the fabric sock in terms of transfer stress and strain, in both the circumferential and longitudinal directions. Accordingly, another aspect of the invention is a method of using mathematical modeling to predict the suitable requirements for a non-linear response in prosthesis, and optimizing the design parameters for a portion of the prosthesis, matched to a particular host anatomy.

### BRIEF DESCRIPTION OF THE DRAWINGS

FIG. 1 is a perspective view of a first embodiment vascular graft prosthesis.

FIG. 2 is a schematic sectional view of the anatomy of a vascular wall.

FIG. 3 is a graph of the non-linear elastic response of a natural artery due to the effect of collagen and elastin in the adventitia.

FIG. 4 is a graph of the change in internal pressure versus diameter change showing non-linear adventitial effect on a porous structure.

FIG. 5 is a graph showing the non-linear exponential stress-strain characteristics for adventitial structures in both the circumferential and longitudinal directions.

FIG. 6 is a static schematic section of a geometric fabric construction.

FIG. 7 is the material of FIG. 6 under stress and strain loading.

FIG. 8 is a graph of a representative stress-strain non-linear curve desired of fabric according to the invention corresponding to that shown in FIGS. 6 and 7.

FIG. 9 is a first static schematic view of a two-material fabric construction.

FIG. 10 is the material of FIG. 9 under stress and strain loading

FIG. 11 is a variation of the two-material fabric construction.

FIG. 12 is the material of FIG. 11 under stress and strain loading.

FIG. 13 is a perspective view of a second embodiment vascular graft prosthesis.

FIG. 14 is a perspective view of a third embodiment vascular graft prosthesis.

FIG. 15 is a flow chart of a computer-implemented process of the graft design optimization sequence.

FIG. 16 is a flow chart of a computer-implemented process of the graft design optimization sequence.

FIG. 17 is a perspective view of a third embodiment vascular graft prosthesis.

### DETAILED DESCRIPTION OF THE PRESENTLY PREFERRED EMBODIMENTS

Various techniques to provide prosthetic vascular grafts have been provided, including, for example, those disclosed in international publication numbers PCT/US97/27629, PCT/US99/27504, PCT/US99/27629, all commonly owned by the current assignee, and which are all incorporated herein by reference. These disclosures, and others, articulate means by which a porous scaffold may be utilized to promote tissue ingrowth as part of the implanting and patentcy of a vascular graft.

One of said applications discloses an improved prosthetic vascular graft, which is created with a synthetic scaffold of transmural ingrowth channels, which are characterized by continuous, uninterrupted, well-defined dimension. A simulated cell structure with unit cells approximating pentagonal dodecahedrons allows such channels to be formed. A unit cell created in a foam type structure can be, and often is, represented by an idealized pentagonal dodecahedron. The process for producing such well-defined pores (i.e., voids) in a synthetic scaffold can be achieved using spherical, soluble micro beads as an extractable filler.

Another teaching in the above references includes a foam-type vascular prosthesis (including structures such vascular grafts, heart valves, sewing rings, and other vessels) with well-defined angio-permissive open porosity. In at least one embodiment, the invention consists of a tubular vascular graft for small diameter applications (2-6 mm ID) containing well defined (essentially spherical; 10-300 μm diameter), interconnected (interconnecting window diameter=30-70% of the pore diameter) pores that would allow for the uninterrupted transmural ingrowth of tissue (from the ablumenal to the lumenal surface). In order for the graft to simulate the elasticity of natural blood vessels, the materials of choice are: polymers, including synthetic polymers, elastomers, and polyurethanes. Examples include products manufactured under the trade names of Vialon, Cardiomat, Erythrothane, Renathane, Tecoplast, Biomer, Mitrathane, Cardiothane, Rimplast, Pursil, Carbosil.

Another teaching in the above references includes porous synthetic grafts with oriented angio-permissive open porosity ingrowth channels. A foam-type structure is disclosed which comprises interconnected spheroid voids, that would not only allow for the uninterrupted ingrowth of tissue, but also allow for the circumferential orientation of the ingrowing/ingrown tissue in sympathy to the pulsatile expansion of the structure to be emulated. Preferably, the ingrowth channels physically confine ingrowth in the desired directions. Radial interconnections between successive helical channels (where the channels “cross”), allow for both radial and circumferential ingrowth.

A further teaching in the above references includes a transmural concentric multi-layer ingrowth matrix with well-defined porosity. Grafts which are made of a foam type (or containing helically oriented pores) are filled with an ingrowth matrix that facilitates graft healing by allowing preferential ingrowth of desired cell-types preferentially over unwanted types. These matrices are hydrogels that the cells are able to degrade during their penetration into the graft wall. The hydrogels are either of synthetic origin (polyethylene glycol (PEG), etc.) or of biologic origin (proteins, polysaccharides)). The matrices may also contain growth factors or genes that can produce the growth factors. The method of incorporating the growth factors is also important. Although simple inclusion (admixture) is claimed, different ways of attaching the growth factors to the matrix in order to provide a slow release are disclosed. In addition to growth factors, other pro-angiogenic and anti-apoptotic substances are incorporated into the gels. In the case of PEG gels, both adhesive and degradable peptide sequences are required to allow the cells to adhere to the gel, and to allow the ingrowing cells to degrade and migrate through the gel. The correct choice of adhesive and degradable sequences allows for the susceptibility of the gel towards ingrowth by particular cell types.

In the above descriptions, the gel is said to fill the entire porous structure. However, in another embodiment of the technology, a gradient of ingrowth matrix material is within the pores, e.g. one formulation is on the outer “edge” of a pore, with a gradient toward another formulation in the center of the pore. Another embodiment may be similar, but with discrete layers (onion type concentric layers in the case of spherical pores; concentric tubular layers for oriented channel porosity) instead of a gradient. In the embodiments, each formulation or layer is optimized for specific cell types. This would, for example, allow for the preferential ingrowth of endothelial cells in the middle of a pore with smooth muscle cells growing in a concentric layer around the endothelial cells, as happens in naturally occurring angiogenesis.

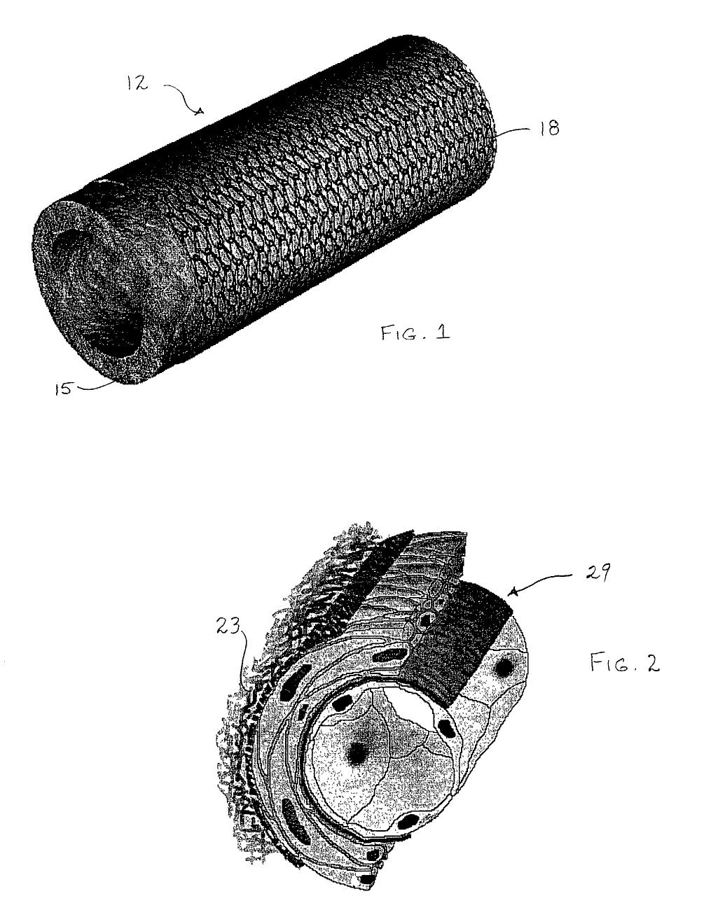

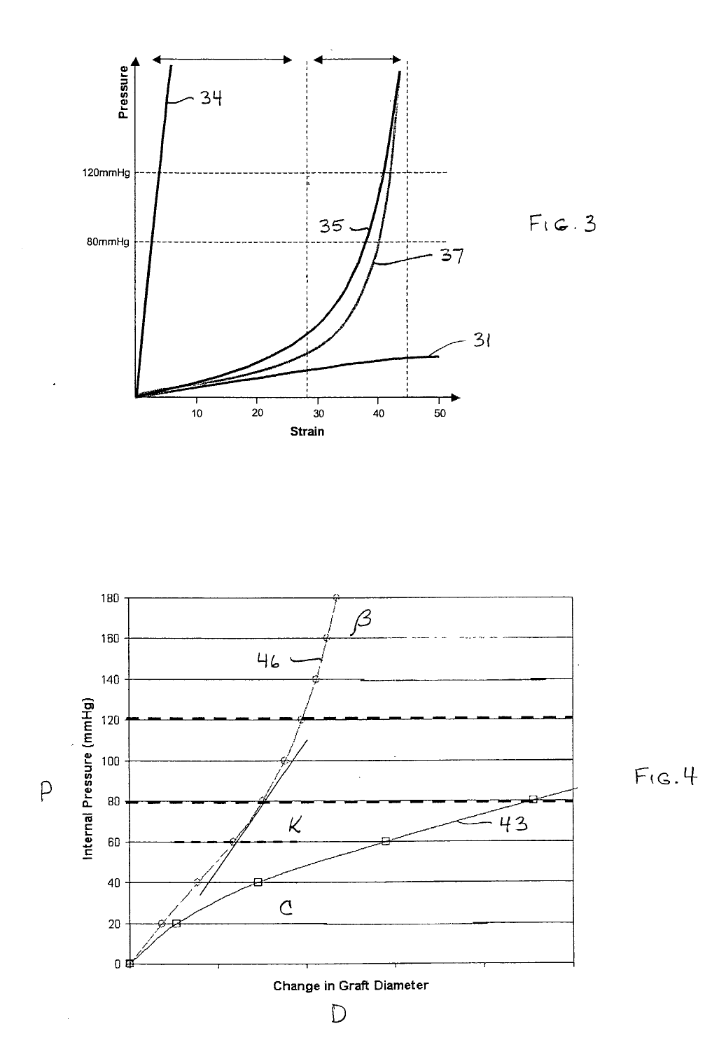

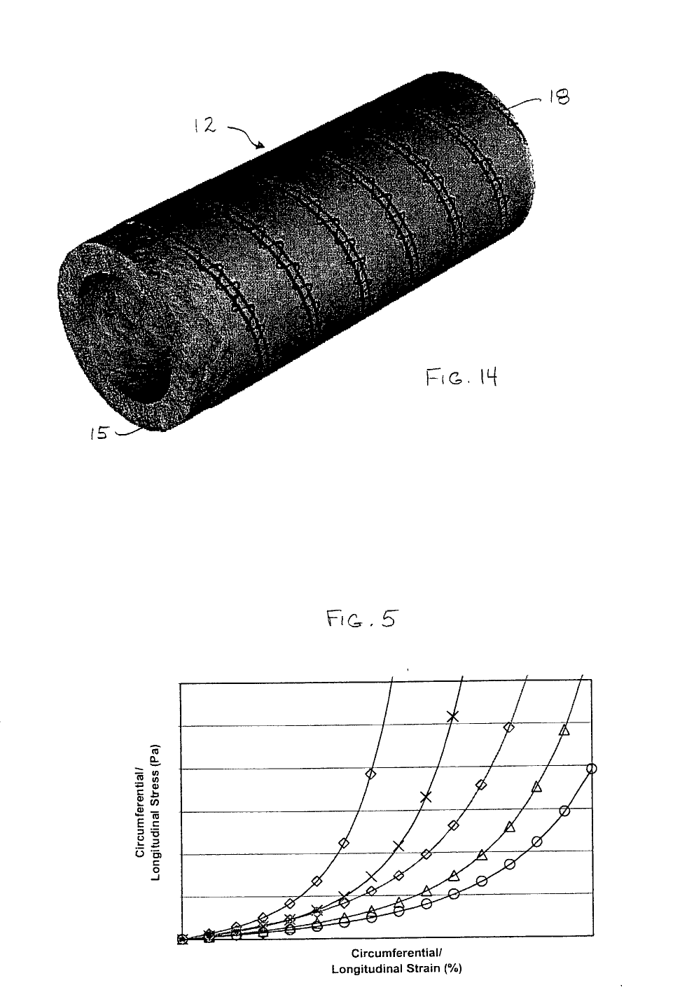

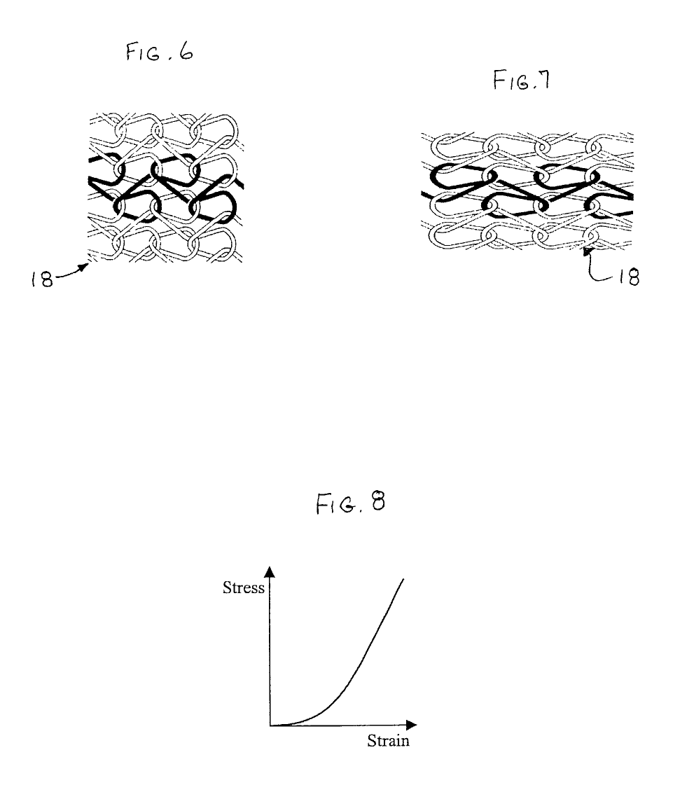

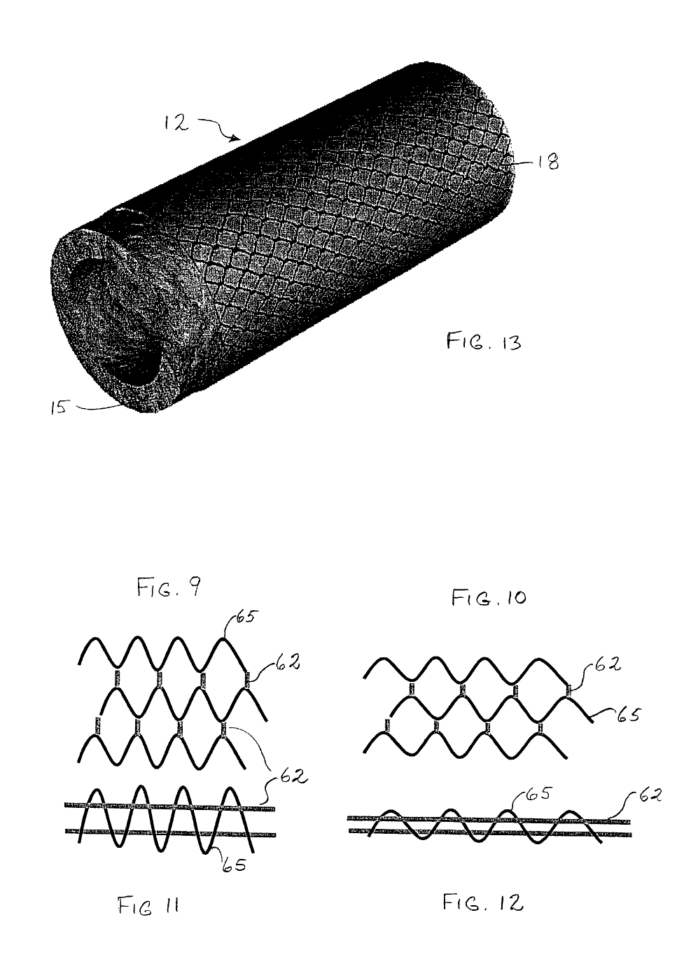

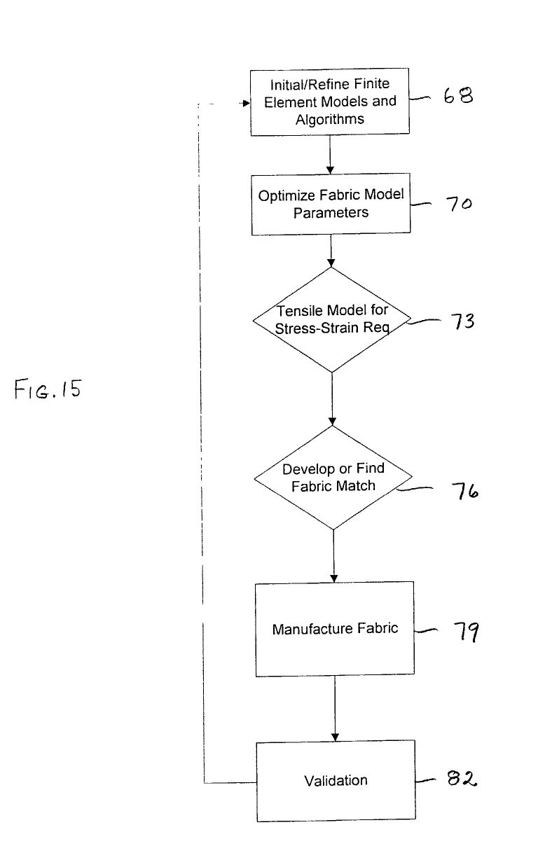

Various methods for producing porous graft materials having well defined pores in the graft tubular wall result in excellent prosthetic materials. However, such materials have heretofore been disclosed and/or rendered without the further benefits and advantages of certain additional structure to optimize the match of the graft material with the host tissue. FIG. 1 illustrates one embodiment of prosthetic vascular graft 12 having a substructure 15 (also referred to herein as an inner material layer, an inner matrix or tube structure, the porous substructure, or simply the graft inner material 15) and an adventitial structure 18 (also referred to herein as outer or reinforcing layer, a reinforcing sock/structure, adventitial layer or fabric reinforcing layer/structure 18). Inner material layer 15 (which may be porous, super porous or highly porous) provides a scaffold that permits unrestricted cellular ingrowth and healing. Inner material 15 is made from any of a plurality of materials, including, for example, those noted above. However, other biocompatible materials having the appropriate material characteristics may be utilized according to the inventions herein. Examples of additional materials for consideration include but are not limited to those in Table I.

TABLE I

| Material Type | Designation/Trade Name | C_{d} |
|---|---|---|
| Polyurethane | Micro porous foam | 12.3 |
| Biomer | Micro porous foam | 49.5 |
| Polyurethane/Siloxane | Micro porous foam | 13.2 |
| Copolymer | | |
| Biomer | Micro porous foam | 13.5-10.3 |
| Estane 5714 | Micro porous foam | 230% dilation |
| | | (6 months) |
| Segmented polyurethane | — | 9.7 |
| PU/MDI/PC/DO/EDA | Chronoflex graft | 8.1 |

The adventitial fabric reinforcing structure 18 permits the prosthesis 12 to exhibit the characteristic non-linear mechanical properties of a natural host blood vessel. As will be discussed and shown below, the unique adventitial fabric structure when combined with the inner tube structure mimics the non-linear mechanical properties of the host vessel. Accordingly, it should be noted that the reference to “adventitial” is a functional (rather than merely location) term. As will be shown below, embodiments of the invention may include fabric reinforcing layer 18 between two layers of material having the characteristics of the above inner material layer 15, or somehow otherwise integrated into a porous or inner layer of material designed to promote tissue ingrowth. In combination, the fabric sock type of prosthesis 12 (and alternate embodiments herein) allows for the unrestricted growth of tissue into the porous substructure due to its highly porous nature, and insures the combined composite structure prevents over dilatation of the vascular graft.

Various attempts to provide compliant grafts consistent with healing properties have resulted in combinations of woven, knit, wound, polymer, extrusion and molding techniques being used. These various methods have provided for either healing properties or compliance. However, these methods have decidedly not provided a graft that actually mimics the non-linear elastic response of a natural artery of a host vessel while simultaneously providing the healing and compliance characteristics of a natural vessel. Designers of prior vascular grafts have failed to recognize the importance of this non-linear elastic response of a natural artery or host vessel as an integral requirement for achieving an optimal prosthetic vascular graft. The present invention overcomes this deficiency in the art by developing methods and techniques to demonstrate non-linear mechanical properties similar to that of a natural artery, and methods and techniques to optimize a fabric reinforcing structure to give the required compliance for various pore structures and blood vessels attached to the fabric structure. The invention further provides the enhanced self-healing abilities of the composite graft by utilizing the porous structure to promote uninterrupted cellular ingrowth and vascularization of the porous substructure. The result is a highly patent graft, which insures against over dilatation at higher blood pressures through use of non-linear stiffening characteristics, as further described herein below. Such a bi-structured or bi-layered system, i.e., one consisting of a porous inner tube or layer and an outer fabric reinforcing layer having non-linear stiffening characteristics, is useful for variously sized prostheses and possibly other elements of the vascular system.

As noted above, inner layer 15 may be manufactured utilizing various techniques, although one preferred technique includes a polymer formed by molding an admixture of polymer, solvent, and spherical, soluble micro beads of a desired diameter. The extraction of the beads and the precipitation of the polymer renders a tubular structure containing well-defined pores in the tube wall suitable for use as a synthetic, vascular graft prosthesis. Fabric reinforcing layer 18 is designed to utilize the geometrical and mechanical properties of either one, two or a plurality of particular material types (although two is preferable) to provide the fabric's non-linear stiffening characteristics.

In one embodiment, the construction of the fabric reinforcing material 18 will be through either a knit, weave or spirally wound mesh or a combination of these to provide this non-linear response. Such non-linear characteristics of the fabric reinforcing material are dependent on and are also determined by the characteristics of the inner substructure. Accordingly, these two structural types, namely the inner structure and the fabric sock structure, will interact in such a way as to provide a dynamic and static non-linear elastic response, where the combined elastic modulus increases exponentially as internal pressure is increased. Again, this dynamic response will mimic the mechanical properties of natural adventitial tissue, which will be further discussed herein below.

FIG. 2 is a sectional representation of vascular tissue useful for illustrating the relation of the natural vessel structure with the prosthetic vascular graft structure of the invention. The natural adventitial layer 23 of an artery 29 is comprised of two main tissue types that contribute to the mechanical properties of the natural artery, namely elastin and collagen. The mechanical properties of these two soft tissue components are described in Table II below:

TABLE II

| Soft Tissue | Elastic Modulus (Pa) | Max Strain (%) |
|---|---|---|
| Elastin | 4 × 10⁵ | 130 |
| Collagen | 1 × 10⁹ | 2-4 |

As shown in the above table, the two soft tissue types have a large difference in mechanical properties, with one being very elastic (elastin) and the other being very stiff (collagen). These two tissue types combine in the adventitial layer to produce a non-linear elastic response. As shown in FIG. 3, the combined effect of the characteristics of elastin 31 and collagen 34 (only playing a role at higher strains) results in a non-linear response curve (shown in loading 35 and off loading 37 configurations) within the physiological range of a natural artery between about 80-120 mm Hg. This characteristic of pulsatile expansion of arteries requires excellent mechanical compliance of any prosthetic graft, i.e., a close mimicking by the prosthetic article of the way in which the natural vessel distends under change in blood pressure.

Compliance is the measure of diameter change with pressure, and may be determined by the formulas shown below. The relevant change in volumes, diameters and pressures refer to the change between systolic and diastolic values. These formulations can be calculated in a dynamic situation under quasi-static/static conditions, and are thus referred to as dynamic and static compliance respectively. Dynamic diameter compliance is a preferred value for reference.

$${C}_{v} = \frac{Δ V}{{V}_{diastolic} Δ P} \times 100 \times 100 \text{mmHg}$$

for volume; and

$${C}_{d} = \frac{Δ D}{{D}_{diastolic} Δ P} \times 100 \times 100 \text{mmHg}$$

for diameter/radial.

The stiffness of blood vessels is stated as a Stiffness Index (β), and is a measure of the changes of curvature and diameter, stated as:

$$ln ( \frac{{P}_{systolic}}{{P}_{diastolic}} ) = β ( \frac{{D}_{systolic} - {D}_{diastolic}}{{D}_{diastolic}} )$$

A related characteristic of blood vessels is that of elastic modulus (K), which is considered a measure of strength, and is stated as:

$$K = \frac{{V}_{diastolic} Δ P}{Δ V} = \frac{{D}_{diastolic} Δ P}{Δ D} ∝ \frac{1}{C}$$

Recognition of the relationships among the mechanical components of vessels is essential to the design of prosthetic grafts having proper compliance values for the recipient patient. FIG. 4 shows that, essentially, Compliance (C) is proportional to the inverse of the slope at a particular diameter (D). The Elastic Modulus (K) is proportional to the slope of the curve at a particular diameter (D); and the Stiffness Index (β) is related to the log of the curve and is a constant.

Compliance data (C_{d}) of natural vessels of humans is known by vessel type and by age of the vessel (i.e., age of patient). For example, a common carotid artery has about a 6.6%/100 mm Hg compliance value. The values for a superficial femoral artery and a femoral artery are 1.8%/100 mm Hg and 5.4%/100 mm Hg, respectively. A value for a saphenous vein, however, is about 4.4%/100 mm Hg, while an aorta ranges from about 13.0-20.0%/100 mm Hg, depending on the location. Also, the lengths of bypass grafts according to location in the body must also be considered, and quite allot of variance is encountered. It is also known that the diameter of various arteries change over both short and long periods of time, and this has a particularly significant impact on overall compliance values (C). It is therefore quite important to properly mimic the compliance value of a natural vessel with that of the synthetic grafts being used in place of the natural vessel.

The success of a vascular prosthesis is dependent in part, on the matching of the mechanical behavior of the implant with that of the native vessel. Of the various mechanical characteristics of arteries, compliance is considered the most important factor, which in-turn influences blood flow and pressure distribution along the vascular arterial tree. Compliance is the degree to which the vessel distends under change in blood pressure during the cardiac cycle. Research has shown that compliance matching between the implant and the native vessel is an important factor in determining the success of the prosthetic graft.

The success of a new graft also depends on the in-growth of tissue. The graft therefore has to be sufficiently porous to allow for this in-growth. However, the greater the porosity the lower the mechanical strength and the higher the compliance. Therefore, much of the modeling to create prosthetic grafts is concerned with balancing the graft porosity (and hence the in-growth space) with mechanical strength and compliance. The materials used for the graft construction are time-dependant and therefore the graft requires wall reinforcement to prevent long-term dilation of the vessel. Thus a composite reinforcement structure has to be designed.

Therefore, a goal of the adventitial fabric reinforcing sock 18 is to utilize either the mechanical characteristics of two individual materials with similar mechanical properties as that of elastin and collagen (i.e., one elastic and the other stiff) or to utilize geometrical properties (i.e., wavy, knit constructions) to produce a non-linear elastic response as shown by curves 35 and 37 in FIG. 3. This non-linear elastic response may be achieved by loosely attaching or enveloping the stiff material to or around the elastic material, whereby, when the combined material is stretched the elastic material takes the initial strain and the stiff material starts to unbundle. Once the stiff material has unbundled, then the strain is gradually taken up by the stiff material, which produces the non-linear elastic response-, which is similar to the inter-relationship of the elastin and collagen in the natural adventitial structure. Similarly, with the geometrical properties of textiles, when the fibers/yarns are fully aligned then the fabric takes up all the stress. Examples of these techniques will be disclosed below.

FIG. 4 shows some of the advantages of achieving the characteristics of the adventitial fabric-reinforcing layer. In particular, FIG. 4 illustrates the change in internal pressure (P) within the vascular structure versus the change in diameter (D) of the vascular graft for both an inner substructure without adventitial support, shown as line 43, and the porous substructure with adventitial fabric reinforcing, shown at line 46. The changes in internal pressure versus internal diameter change of the graft structure, i.e., the static compliance, illustrates dramatically the stiffening effect provided to the composite structure as a result of the fabric reinforcing layer 18. FIG. 4 also illustrates the difference in graft diameter for identical pressures between the two graft structures represented by lines 43 and 46, and the attendant risk of over dilatation of a vascular graft/porous substrate without adventitial support as an outer layer. Recognition of this substantial effect, and that the non-linear uniaxial and biaxial stress strain responses are of an exponential form, permits the non-linear response of the fabric sock to be calculated for the individual mechanical and geometrical properties of a particular inner structure. These calculations are based on computational, analytical and experimental methods, which will be further discussed below. Stated differently, in view of the mechanical properties of this adventitial structure being dependent on the particular porous substructure characteristics, then these mechanical properties will be described by an exponential curve for both the circumferential and longitudinal (warp and weft) directions similar to those illustrated in FIG. 5 for various selected structures. In this figure, the point of inflection at which the curve changes to non-linear demonstrates different fabric reinforcing phenomena which are configurable using the inventions herein as needed for a specific vessel graft.

One embodiment of an adventitial sock 18 will be generally thinner than the porous or inner substructure 15, having a thickness of between 20 micrometers and 1.0 millimeter. The spacing or passages through the adventitial structure will be large enough to allow for the un-interrupted tissue ingrowth into or through the porous substructure, with the spacing being between approximately 100 μm -3.0 mm and having a diameter of between 2.0-8.0 mm. This adventitial structure will be quite porous and of a similar construction to a net.

At least three different methods of manufacturing the adventitial fabric-reinforcing sock 18 are disclosed. The first method comprises a geometric construction of a fabric, using weave, braid or knit textile constructions, as shown in FIGS. 6, 9, and 11. FIG. 7 is the material of FIG. 6 shown under stress and strain loading.

A corresponding representative stress versus strain curve is shown in FIG. 8. These examples include such textile structures as knits, weaves and braided structures. The woven pattern will likely be in a tubular form, and will likely be of fine construction and extremely porous. The fabric reinforcing layer 18 will be connected to the super porous layer 15 either by imbedding it within or placing it on the layer 15, or loosely attaching it to the porous structure's surface. Another embodiment for attaching includes connecting the adventitial layer 18 fabric at pre-defined or various points along the length of the porous structure layer 15. The first method uses the non-linear stiffening properties of textile fabrics unique to their geometrical construction or the use of two or more, mechanically different, yarn types.

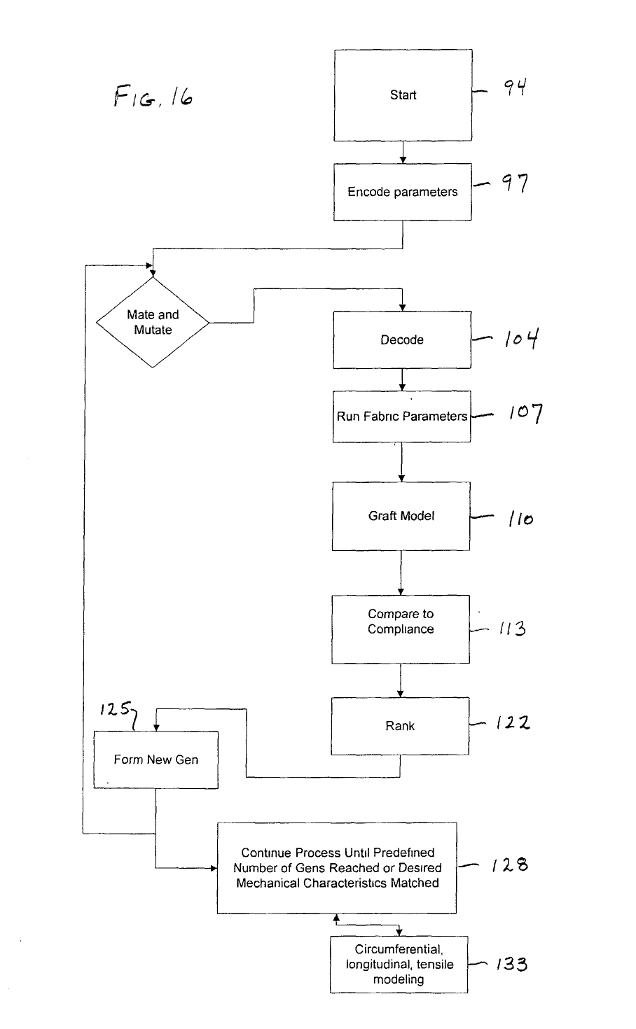

A second method comprises a hybrid composite tubular mesh structure using two particular material types, i.e., stiff and elastic, as shown in FIG. 13. In this figure, adventitial mesh 18 is made of two particular material types as shown in FIG. 9. The third method utilizes a composite wound structure using two particular material types, i.e. stiff and elastic—such as that shown in FIG. 14 in which wound material 18 is made of two particular material types as shown in FIG. 11.

A third method comprises placing a spirally wound mesh consisting of two particular material fiber types (elastic 62 and stiff 65) around or within the porous structure, as illustrated in FIGS. 9 through 12. The pitch and angle of the windings may be changed to achieve the desired adventitial properties and multiple combinations of pitch, number of winds and orientation may be used successfully. The spiral wound structure or adventitial mesh 18 may be attached to the porous structure 15 loosely or at intervals along its length or it may also be an internal part of structure 15. In addition, various combinations of the attachment techniques for attaching the adventitial fabric to the porous structure are possible. In one embodiment, the materials will be attached in a circumferential fashion along a preferred orientation for load bearing purposes of between 0-10 degrees pitch along the graft's length.

FIG. 1 illustrated graft 12 with adventitial fabric 18 formed with the geometrical properties embodiment to achieve non-linear characteristics. FIG. 13 is an example of a mesh type structure, which uses a mesh similar to that shown in FIG. 9, and includes bi-layered graft 12, with highly porous sub-structure 15, and adventitial layer 18. However, the graft of FIG. 13 uses a material or layer 18 having the two-material properties embodiment (elastic and stiff) for achieving the non-linear characteristics. As such, the embodiment of FIG. 1 may be referred to as a fabric reinforced structure for layer 18, and FIG. 13 may be referred to as a mesh reinforced structure for layer 18. Similarly, FIG. 14 illustrates a wound reinforced structure for layer 18 comprising two material properties wound together as shown.

As structures have become increasingly complex, not only in design but also in the range of material use, pure analytical methods have begun to fail in describing the behavior of such structures. Due to the scientific challenge of precisely matching a vascular graft of the type described herein to a host, analytical methods are rendered obsolete. Development of a highly porous prosthetic vascular graft, which allows unrestricted vessel ingrowth from surrounding tissue, as well as a graft which matches the mechanical requirements and dynamic compliance similar to that of a host is made possible, however, with advanced mathematical techniques. In particular, the use of numerical modeling with such tools as, for example, Finite Element Models and Methods, relying on continuum mechanics, along with certain other tools makes this level of customization feasible.

It is necessary to develop a fabric constitutive relation (for the fabric 18) and implement it in a general purpose numerical package. Then the designer must develop an optimization routine, which can interact with the numerical package and optimize the fabric model parameters for specific criteria. The developed fabric constitutive relation and optimization routine is used in a graft numerical model to optimize the fabric model parameters, which in turn produces an external fabric-reinforced graft having a defined dynamic diameter compliance, non-linear characteristic. In one example, this dynamic diameter compliance is selected at 6% /100 mm Hg, to mimic certain human vessels. The optimized fabric parameters are then utilized to find the transverse mechanical requirements of the fabric, by implementing the fabric model in a tensile Finite Element Model.

Other additional developmental steps may include correlation of the results obtained from the Finite Element Models against experimental data. This entire process, using computer implemented steps, equipment, software and processes, aids in the development of an optimum fabric for use with a custom graft in a host patient.

It is known that the stress-strain relation of a fabric is highly non-linear in the low stress region and then becomes linear after a critical point. The critical point varies from fabric to fabric, and the various deformation modes. Indeed, in one embodiment, a manufacturing step includes longitudinal or other pre-straining of the adventitial outer material over the graft material. In one embodiment, the circumferential pre-straining on the adventitial sock over the inner porous structure is performed so that when released the sock will contract over the inner structure under no-load conditions. This circumferentially pre-stressed example mimics the condition found in a natural blood vessel. These and other characteristics have been modeled for various reasons. However, the compliance matching and non-linear mechanics problems faced by the present inventors has so many variables as to require a new design process. Accordingly, the combination of numerical modeling (e.g., graft model, circumferential or longitudinal, and tensile models), numerical algorithms (including but not limited to Genetic Algorithms (e.g., GA1 and GA2)), and/or various other optimization techniques have been developed. In general, optimization techniques are a useful tool in finding the best solution to maximize/minimize criteria, such as weight, strength and dimension. Traditional optimization techniques, known as hill climbing techniques, require relations between the variables and the parameter to be optimized. These relations normally take the form of differentials. If these relations are linear then these problems can be solved directly. However, iterative searches normally have to be used to find the solutions where they are nonlinear. One such iterative scheme is nonlinear regression. However, these nonlinear traditional hill climbing techniques have been found to be problematic when three or more non-linearly-related variables are solved for, or when the relations between the variables cannot be explicitly defined. This has led to new methods of optimization such as those offered by Genetic Algorithms.

Genetic Algorithms do not require explicit differential relations between the variables. Instead they require a single objective function, which describes whether a set of parameters is converging on an optimal solution. This therefore makes Genetic Algorithms a powerful and useful optimization tool where relations between the variables cannot be defined. A Genetic Algorithm is an optimization routine, which randomly utilizes a certain group of parameters (e.g., a chromosome) that run in a model and optimizes these for a certain objective function. A generation (a number of set parameters) is used to obtain values from the objective function. These generation members are then ranked accordingly, where the best solutions found are used to produce a new generation through the process of crossover (mating two generation members to give a new member) and mutation (changing a generation member randomly). The new generation members are then used in the objective function and the process is repeated. Genetic Algorithms are based heavily on genetic work and the principles of nature and reproduction, hence the concepts of generation, mutation, crossover (mate) and chromosome.

Genetic Algorithms have known uses for unconstrained optimization problems. However, many engineering problems are highly constrained and nonlinear, which results in a complex search space with regions of feasibility and infeasibility. Constraints can be classified into two categories, explicit and implicit constraints. Explicit constraints are those, which can be checked without finding a solution. An example is the value of a design variable, which has a maximum value constraint. Implicit constraints are those that can only be checked once a solution is found. An example of this would be where the result of the solution affects a parameter.

One of the benefits of using Genetic Algorithms is that they are unlikely to become trapped at a local maximum. Since Genetic Algorithms do not need derivative information, the relations between the variables and the objective function are not required. A Genetic Algorithm is not path dependent and therefore does not fall pray to its initial starting point. Genetic Algorithms are able to work in domains that are discontinuous, ill-defined or have many local maximums. Indeed, Genetic Algorithms are particularly well suited to searching large, complicated and unpredictable search spaces. The parallel nature of Genetic Algorithms (i.e., their ability to search a number of solutions at one time) is also an advantage. Another advantage of Genetic Algorithms over traditional methods is their ability to maximize their search capabilities by introducing mutations into certain generations. Although the Genetic Algorithm seems robust, it does have the problem of being computationally expensive and therefore generally takes longer to converge on a solution. However, its advantages as a general purpose optimization tool capable of solving many variables that are multidimensional, discontinuous and nonlinear, far outweigh its drawback of computational expense. Thus a Genetic Algorithm has potential in the medical field, where, the number of variables is high, extremely nonlinear, not well-defined and difficult to relate by way of differential relations.

In one embodiment, a graft with adventitial fabric must be modeled mathematically using a Finite Element package to model and analyze the graft design for various fabric reinforcing behavior. Then, using an optimization technique such as (but not limited to) a Genetic Algorithm, these fabric parameters are adjusted until an optimal solution is found, giving a desired dynamic diameter compliance for various porous structures. The optimized solutions found are then utilized in tensile Finite Element Models to obtain transverse stress-strain characteristics of the fabric for uniaxial/biaxial, or longitudinal and circumferential, tests. As is known in Finite Element Modeling, the constitutive relations or material characteristics must first be determined and entered via user material subroutines.

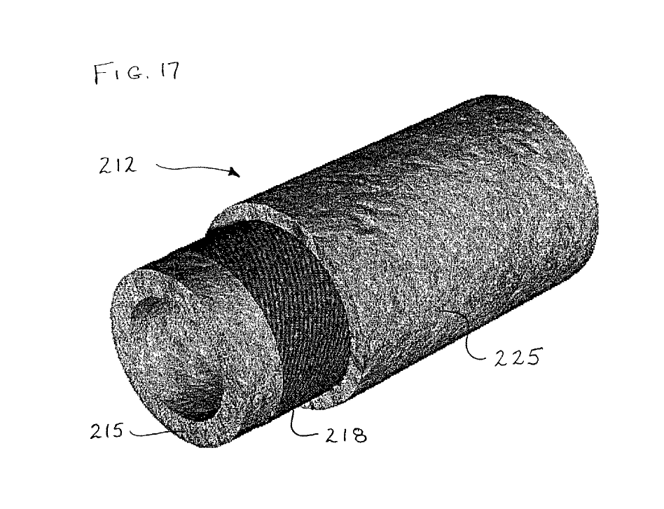

In view of the stress-strain characteristics of fabrics and soft tissue being similar, a model that is used for general non-linear anisotropic soft-tissue is used to also describe the material behavior of the fabric of this invention. FIG. 15 illustrates the use of a Finite Element Model process; generally as described above, also using further numerical modeling such as with an exemplary Genetic Algorithm technique- although other algorithms are useful in this function in varying scope. In the process exemplified in FIG. 15, step 68 provides initial or refined finite element models and further algorithms, such as Genetic Algorithms. Step 70 then optimizes the fabric model parameters until a desired stress-strain requirement is met, such as in one embodiment when a 6%/100 mm Hg compliance is achieved. Step 73 then uses the optimized fabric parameters in a tensile model for stress-strain requirements to develop or find fabric with the same stress-strain behavior at step 76. The fabric is manufactured at step 79, and then physically tested at step 82 for model validation. Finite Element Models and Genetic Algorithms are generated and then again refined, as appropriate, at step 68. The basis of the process is to find and mimic the requirements of tissue in a fabric, while ensuring that the porous structure of the overall prosthetic material promotes tissue ingrowth, has a proper compliance value, and is structurally strong.

In one embodiment, a scripting routine known as a Perl® scripting routine, was utilized to run a Genetic Algorithm which optimized an objective function based on the results of Finite Element analysis. Within the scripting, a Genetic Algorithm (“GA”) was utilized, and Finite Element Models were written and run on ABAQUS, version 5-8.8, a commercially available Finite Element package. The results were then read and utilized in the Genetic Algorithm's objective function. The process was repeated until the desired results were obtained or termination after a pre-determined number of generations.

Two GAs were utilized, namely GA1 and GA2. The first, GA1, optimized the fabric model parameters to obtain the desired dynamic diameter compliance or static compliance from a dynamic or static compliance model. The best parameter results obtained from the GA were then utilized in the tensile model to obtain a range of transverse uniaxial and biaxial stress-strain curves. The second, GA2, optimized the fabric model parameters to obtain the transverse uniaxial stress-strain curves for a number of fabrics physically tested. This GA ran two uniaxial tensile test models for a single set of fabric parameters to obtain stress-strain curves in certain, for example transverse, directions. These parameters were then optimized until they matched the physical results for each fabric. GA2 is utilized to obtain some comparative data for the modeling process and to identify the ability of the exponential fabric model to describe the transverse tensile behavior of fabrics.

As shown in FIG. 16, another embodiment of this process and method commences at step 94 by starting with fabric model parameters that run in models, such as the ABAQUS models. Step 97 requires encoding the model parameters into (mathematical) chromosomes. The mating or crossing and mutating of the chromosomes to form generation members is accomplished at step 101, with the chromosomes then being decoded to fabric parameters in step 104. Step 107 runs the fabric parameters in the models, at GA1 in steps 110 and 113. At step 122 the generation members are ranked so that either the two best members may be used at step 125 to form a new generation or a decision may be made on the desired number of best solutions to be kept to help produce the next generation. As shown in step 128 the process continues until a predefined number of generations is reached or the desired mechanical characteristics are met, e.g., 30 generations in one embodiment or about 6%/100 mmHg dynamic compliance is obtained to within a certain tolerance. Then at step 133 there is circumferential, longitudinal and tensile modeling to obtain reinforcing requirements.

Various optimization techniques and fitness functions are employable within this process. For example, a time dependent model for tissue growth may be incorporated in the fabric constitutive relation to compensate for the physical behavior of the fabric inside the host and while experiencing the effects on its mechanical characteristics, e.g. more stiffness or other mechanical changes. For example, one embodiment predicts the mechanical performance changes over time due to projected tissue ingrowth into the graft material as well as predicting or modeling the degradation of portions of the prosthesis material (and the adventitial material in particular) during the tissue ingrowth process. This may be done at least in part by using a gel or the like to simulate ingrowth or by pre-clotting to achieve the desired simulation state. Another example of optimization techniques includes using mesh sensitivity studies to accommodate stress variations on the fabric at different locations on the graft. It should be recognized that the accuracy of the design process depends on the quality of the approximation of the constitutive relations used to describe the materials and the detail to which the finite element models equal the physical situation. Accordingly, in addition to compliance-related variables, it may be useful to incorporate longitudinal strain requirements, maximum allowable compression seen through the porous structure, and systolic and diastolic diameter requirements. For example, these requirements could be included into the objective function of numerical models and solutions found which optimize each of many different scenarios for one or more host patients.

Additional processes to improve the design of the optimum prosthesis according to this invention may include (for the fabric constitutive model): a thorough investigation into a general fabric strain energy function which would include viscoelastic and plastic effects; incorporation of the effects of tissue in-growth under physiological conditions on the fabric's and porous polymer's mechanical responses; and use of a model which pre-stresses the fabric around the porous structure in a way which achieves the desired non-linearity of the fabric stress-strain curve. Additional processes to improve the design of the optimum prosthesis according to this invention may include (for the numerical algorithms and optimizations): utilization of a biaxial tensile Finite Element Model for finding fabric parameters so as to ensure that the analysis does not need to include the Poisson's effect in the fitness function and shear properties will be neglected; utilization of a non-linear regression technique to solve for some of the fabric strain energy parameters; adding in new sections to the fitness function such as a system of elimination and change of the penalty functions over time; finding the optimal thickness of the porous structure to ensure that minimal compression is seen through the porous wall, thus increasing the polymers' in-growth abilities; improving consistency in porous structure formation and graft circumferential mechanics; and optimizing the pre-stressing of the fabric before placing it around the porous structure, to give it a lower point of inflection for the static compliance curves. Additional processes to improve the design of the optimum prosthesis according to this invention may include (for the graft manufacture): development of tubular fabrics which do not require sewing; and development of further methods of attaching the fabric to the porous structure, including possibly molding the fabric into the porous structure.

What is provided therefore is a product which is made by one of several processes, and which is also made to a customized compliance value per patient need, if desired. In particular, a computer-implemented method is used to design a vascular graft prosthesis having desired mechanical characteristics, which mimic the characteristics of natural vessels. The steps of this method include providing software implemented means for entering parameters of fabric graft material and graft data into an encoding processor, and then entering such data; implementing a plurality of computer implemented optimization algorithms which implement a numerical composite graft model analysis and numerical composite circumferential and longitudinal tensile model analyses on a number of parameters. Then new data generations are formed using the optimization algorithms performing iterations until desired mechanical characteristics are achieved.

Utilization of this and related design processes disclosed herein represents a remarkable innovation which allows manufacturing of a vascular graft prosthesis in which an improvement comprises having an adventitial material controlling and interacting with an inner graft structure, wherein the adventitial material is more elastic and less stiff than the inner graft structure material and is characterized by a non-linear elastic response which mimics the natural vessel of the host. As such, Applicants have now identified new and improved mechanisms by which the technical means of harnessing the ongoing and simultaneous revolutions in medical device technologies, information technologies, modeling and design software and algorithms, and patient population healthcare management techniques achieves a new level of result. In particular, these technical means result in improved patient care and long term graft/vessel patency, less rejection of implanted grafts and other prostheses, and medical devices which are customized to individual patient needs. This business technical methodology has enabled improved quality of care; and improved efficiencies and economics are a further consequence. As shown herein, the technical effect of the selected protocols and steps in the design and manufacturing processes has resulted in a significant technical contribution to the art of vascular prostheses. This is also a pioneering technical business effort in identifying historic problems, and applying technical methodology to combine the best design and manufacturing technologies to improve the care of patients with vascular disease or conditions requiring replacement grafts. On at least one level, the contributions of this invention enable economic functionality to attach to highly innovative technical solutions in value added formats.

Examples of products made by these processes, and products and markets created, include the graft prostheses shown in FIGS. 1, 6-7, 9-14, and 17. FIG. 17 illustrates an embodiment of graft prosthesis 212 having a first inner material 215, and second fabric reinforcing or adventitial material 218, and a third material 225 which may have similar tissue ingrowth or other properties as first inner material 215.

While the embodiments of the invention described herein are presently preferred, various modifications and improvements can be made without departing from the spirit and scope of the invention. The scope of the invention is indicated by the appended claims, and all changes that fall within the meaning and range of equivalents are intended to be embraced therein.

We claim:

1. A vascular graft prosthesis having a bi-layer wall structure configured to optimize mechanical compliance to a host vessel, comprising:
an inner material shaped as a tube structure which allows uninterrupted cellular growth; and
an outer adventitial material connected to the inner tube which allows for cellular in-growth, said adventitial material being characterized by a non-linear elastic response.

2. The vascular graft prosthesis of claim 1 in which the outer adventitial material is constructed to enable substantially uninterrupted tissue ingrowth into the inner tube structure.

3. The vascular graft prosthesis of claim 1 in which the inner material is porous.

4. The vascular graft prosthesis of claim 3 wherein said inner porous tube comprises a polymer structure having a wall, and interconnecting shaped pores in the tube wall; wherein porosity is optimized to maximize cellular in-growth.

5. The vascular graft prosthesis of claim 1 wherein said outer adventitial material comprises a fabric structure with interconnecting shaped pores; wherein the fabric is aligned to allow desired directional growth of tissue through the adventitial structure for maximum cellular in-growth.

6. The vascular graft prosthesis of claim 1 in which the outer adventitial material comprises fibers containing controlled release material.

7. The vascular graft prosthesis of claim 1 wherein said inner tube comprises hyper-elastic isotropic material.

8. The vascular graft prosthesis of claim 1 wherein said inner tube comprises weak orthotropic elastic material.

9. The vascular graft prosthesis of claim 1 wherein said adventitial outer material comprises a non-linear anisotropic fabric-reinforcing sock having properties that increase stiffness with strain.

10. The vascular graft prosthesis of claim 1 wherein said adventitial outer material comprises a non-linear transversely isotropic fabric reinforcing material having properties that increase stiffness with strain.

11. The vascular graft prosthesis of claim 1 wherein said adventitial outer material comprises a non-linear transversely orthotropic fabric reinforcing material having properties that increases stiffness with strain.

12. The vascular graft prosthesis of claim 1 wherein said adventitial outer material, when combined with the inner material, is configurable to achieve any desired dynamic diameter compliance.

13. The vascular graft prosthesis of claim 1 wherein said adventitial outer material, when combined with the inner material, is configurable to achieve any desired static diameter compliance.

14. The vascular graft prosthesis of claim 1 wherein said adventitial outer material, when combined with the inner material, is configurable to achieve any desired quasi-static diameter compliance.

15. The vascular graft prosthesis of claim 1 wherein said adventitial outer material, when combined with the inner material, is configurable to achieve a dynamic diameter compliance value of 6%/100 mm Hg.

16. The vascular graft prosthesis of claim 1 wherein said adventitial outer material is thinner than said inner tube structure.

17. The vascular graft prosthesis of claim 1 wherein said adventitial outer material has a thickness in the range of 0.020-1.0 mm.

18. The vascular graft prosthesis of claim 1 wherein said adventitial outer material has pores with an average diameter in a range of 0.1-3.0 mm.

19. The vascular graft prosthesis of claim 1 wherein said adventitial outer material has pores with an average diameter in a range of 0.1-3.0 mm, allowing for un-interrupted tissue growth; and wherein the overall tube diameter is between about 2.0 -8.0 mm.

20. The vascular graft prosthesis of claim 1 wherein the diameter and internal lumen of said prosthesis is optimized for positional and mechanical requirements depending on the location in the human body.

21. The vascular graft prosthesis of claim 1 wherein said outer material is wound around said inner material.

22. The vascular graft prosthesis of claim 1 wherein said outer material is loosely connected to said inner material.

23. The vascular graft prosthesis of claim 1 wherein said outer material is connected at pre-defined points along a length of said inner tube structure.

24. The vascular graft prosthesis of claim 1 wherein said outer material comprises a combination of stiff and elastic material.

25. The vascular graft prosthesis of claim 1 wherein a portion of the adventitial material is biodegradable.

26. The vascular graft prosthesis of claim 1 in which the inner material is selected from polyurethane, a biomer, a polyurethane/Siloxane copolymer, Estane 5714, and segmented polyurethane.

27. The vascular graft prosthesis of claim 1 in which at least one of the inner material and the outer adventitial material comprises ingrowth matrix material.

28. The vascular graft prosthesis of claim 24 wherein said stiff and elastic materials are in contact with each other so that when said elastic material is stretched it takes the initial strain while said stiff material starts to un-bundle, then producing the non-linear elastic response.

29. The vascular graft prosthesis of claim 24 wherein said stiff and elastic materials are combined as a mesh.

30. The vascular graft prosthesis of claim 24 wherein said stiff and elastic materials are combined as a spiral wind.

31. The vascular graft prosthesis of claim 24 wherein said combination of stiff and elastic materials is attached loosely to a surface of said inner tube.

32. The vascular graft prosthesis of claim 24 wherein said combination of stiff and elastic materials is wound around said tube structure.

33. The vascular graft prosthesis of claim 1 wherein said adventitial outer material comprises a geometrical construction of reinforcing textile fabric structure woven together in a tubular form that allows said textile fabric to increase its stiffness with strain.

34. The vascular graft prosthesis of claim 1 wherein said adventitial outer material comprises a geometrical construction of reinforcing textile fabric structure knitted together in a tubular form that allows said textile fabric to increase its stiffness with strain.

35. The vascular graft prosthesis of claim 1 wherein said adventitial outer material comprises a geometrical construction of reinforcing textile fabric structure braided together in a tubular form that allows said textile fabric to increase its stiffness with strain.

36. The vascular graft prosthesis of claim 1 wherein said adventitial material is a textile fabric structure that is woven together as a weave having at least one set of yarns interlaced at angles to each other.

37. The vascular graft prosthesis of claim 1 wherein said adventitial material is a textile fabric structure, and said textile fabric structure is woven together as a knit having inter-meshed loops of yarn in either weft or warp form.

38. The vascular graft prosthesis of claim 1 wherein said adventitial material is a textile fabric structure, and said textile fabric structure is woven together as a braid having a plurality of yarns crossed over each other sequentially.

39. The vascular graft prosthesis of claim 1 wherein said adventitial material is a textile fabric structure, and said textile fabric structure is a non-woven structure formed by needle-felting yarn into said structure.

40. A vascular graft prosthesis having a wall structure configured to optimize mechanical compliance, diameter, non-linear stiffening characteristics and wall compression to a host vessel, comprising:
an inner material shaped as a tubular structure which allows cellular growth; and
an outer adventitial material positioned around the inner material, said adventitial material being characterized by a non-linear elastic response.

41. The vascular graft prosthesis of claim 40 in which the outer adventitial material is constructed to enable substantially uninterrupted tissue ingrowth into the inner tube structure.

42. The vascular graft prosthesis of claim 40 in which the inner material is porous.

43. The vascular graft prosthesis of claim 40 wherein said inner porous tube comprises a polymer structure having a wall, and interconnecting shaped pores in the tube wall; wherein porosity is optimized to maximize cellular in-growth.

44. The vascular graft prosthesis of claim 40 wherein said adventitial outer material comprises a non-linear anisotropic fabric-reinforcing sock having properties that increase stiffness with strain.

45. The vascular graft prosthesis of claim 40 wherein said adventitial outer material comprises a non-linear transversely isotropic fabric reinforcing material having properties that increase stiffness with strain.

46. The vascular graft prosthesis of claim 40 wherein said adventitial outer material comprises a non-linear transversely orthotropic fabric reinforcing material having properties that increases stiffness with strain.

47. The vascular graft prosthesis of claim 40 wherein said adventitial outer material, when combined with the inner material, is configurable to achieve any desired dynamic diameter compliance.

48. The vascular graft prosthesis of claim 40 wherein said adventitial outer material, when combined with the inner material, is configurable to achieve a dynamic diameter compliance value of 6%/100 mm Hg.

49. The vascular graft prosthesis of claim 40 wherein said adventitial outer material has a thickness in the range of 0.02-1.0 mm.

50. The vascular graft prosthesis of claim 40 wherein said adventitial outer material has pores with an average diameter in a range of 0.1-3.0 mm, allowing for un-interrupted tissue growth; and wherein the overall tube diameter is between about 2.0-8.0 mm.

51. A vascular graft prosthesis having a bi-layer wall structure configured to optimize mechanical compliance to a host vessel, comprising:
an inner porous tube comprising a polymer structure having a circumferential wall; and interconnecting generally uniformly shaped pores in the tube wall; wherein porosity is optimized to maximize uninterrupted cellular growth; and
an outer adventitial material connected to the inner porous tube, said adventitial material being characterized by a non-linear elastic response.

52. The vascular graft prosthesis of claim 51 wherein said adventitial outer material comprises a non-linear anisotropic fabric-reinforcing sock having properties that increases stiffness with strain.

53. The vascular graft prosthesis of claim 51 wherein said adventitial outer material, when combined with the porous material, is configurable to achieve any desired dynamic diameter compliance.

54. The vascular graft prosthesis of claim 51 wherein said adventitial outer material, when combined with the porous material, is configurable to achieve a dynamic diameter compliance of 6%/100 mm Hg.

55. A vascular graft prosthesis having a bi-layer wall structure configured to optimize compliance to a host vessel, comprising:
an inner tubular shaped material structurally configured to promote uninterrupted cellular growth; and
an outer adventitial material contiguous to the inner material, said adventitial material comprising a fabric-reinforcing sock having properties that increase stiffness with strain and being characterized by a non-linear elastic response.

56. A vascular graft prosthesis having a bi-layer wall structure configured to optimize mechanical compliance to a host vessel, comprising:
an inner porous tube which allows uninterrupted cellular growth; and
an outer material connected to the inner porous tube, said adventitial material comprising a non-linear anisotropic fabric-reinforcing sock having properties that increase stiffness with strain and being characterized by a non-linear elastic response.

57. The vascular graft prosthesis of claim 56 wherein said outer material, when combined with the porous material, is configurable to achieve any desired dynamic diameter compliance.

58. The vascular graft prosthesis of claim 56 wherein said outer material, when combined with the porous material, is configurable to achieve a dynamic diameter compliance of 6%/100 mm Hg.

59. A vascular graft prosthesis having a wall structure configured to optimize mechanical compliance, diameter, non-linear stiffening characteristics and wall compression to a host vessel, comprising:
an inner material configured to allow cellular ingrowth; and
an outer adventitial material positioned around the inner material, said adventitial material being characterized by a non-linear elastic response, so that the graft prosthesis matches the compliance values of the host vessel.

60. A vascular graft prosthesis having a wall structure configured to optimize mechanical compliance, diameter, non-linear stiffening characteristics and wall compression to a host vessel, comprising:
an inner material configured to facilitate cellular ingrowth; and
an outer adventitial material positioned around the inner material, said adventitial material being characterized by a non-linear elastic response and a structure to allow uninterrupted tissue ingrowth into said inner material, so that the graft prosthesis matches the compliance values of the host vessel.

61. A vascular graft prosthesis having a wall structure configured to optimize mechanical compliance, diameter, non-linear stiffening characteristics and wall compression to a host vessel, comprising:
an inner material configured to facilitate cellular ingrowth; and
an outer adventitial material positioned around the inner material, said adventitial material being characterized by a configurable non-linear elastic response and a structure to allow uninterrupted tissue ingrowth into said inner material so that the graft prosthesis provides compliance values, which match those of the host vessel at the graft location.

62. A vascular graft prosthesis having a wall structure configured to approximate the natural compliance in a host vessel wall, comprising:
an inner material configured to facilitate cellular ingrowth; and
a fabric reinforcing material in contact with the inner material, said fabric reinforcing material having a structure which allows virtually uninterrupted tissue ingrowth into said inner material and which has a non-linear elastic response to stress which approximates the natural compliance values of the host vessel.

63. A vascular graft prosthesis having a wall structure configured to substantially match the natural compliance values to a host vessel, comprising:
an inner material configured to facilitate cellular ingrowth; and
an outer adventitial material positioned around the inner material and having a thickness of between about 0.020-1.0 mm, said adventitial material having a fabric-like structure which allows substantially uninterrupted tissue ingrowth into said inner material and which has an increased stiffness with strain which regulates the prosthesis shape so as to substantially match the compliance values of the host vessel.

64. A vascular graft prosthesis having a wall structure configurable to desired compliance values appropriate for a host vessel, comprising:
an inner material configured to facilitate cellular ingrowth; and
an outer adventitial material positioned around the inner material and having a thickness of between about 0.020-1.0 mm, said adventitial material having a fabric-like structure which forms pores having an average diameter in a range of about 100 μm-3 mm and which allows uninterrupted tissue ingrowth into said inner material; the adventitial material further having a characteristic of increased stiffness with strain which allows for configuring the compliance values of the graft prosthesis to those which are appropriate for a host vessel.

65. A vascular graft prosthesis having a bi-layer wall structure configured to optimize mechanical compliance to a host vessel, comprising:
an inner porous tube comprising a polymer structure having a circumferential wall; and interconnecting uniformly shaped pores in the tube wall; wherein porosity is optimized to maximize uninterrupted cellular growth; and
an adventitial material connected to the inner porous tube, said adventitial material comprising a non-linear anisotropic fabric-reinforcing sock having properties that increase stiffness with strain and being characterized by a non-linear elastic response.

66. The vascular graft prosthesis of claim 65 wherein said adventitial outer material, when combined with the porous material, is configurable to achieve any desired dynamic diameter compliance.

67. The vascular graft prosthesis of claim 65 wherein said adventitial outer material, when combined with the porous material, is configurable to achieve a dynamic diameter compliance of 6%/100 mm Hg.

68. The vascular graft prosthesis of claim 65 wherein said adventitial outer material is thinner than said inner porous tube structure.

69. The vascular graft prosthesis of claim 65 wherein said adventitial outer material has a thickness in the range of 0.020-1.0 mm.

70. The vascular graft prosthesis of claim 65 wherein said adventitial outer material has pores with an average diameter in a range of 0.1-3.0 mm, allowing for un-interrupted tissue growth; and wherein the overall tube diameter is between about 2.0-8.0 mm.

71. The vascular graft prosthesis of claim 65 wherein the diameter of said prosthesis is optimized for position and mechanical requirements depending on the location and age in the human body.

72. The vascular graft prosthesis of claim 65 wherein said outer material is wound around said inner porous tube.

73. The vascular graft prosthesis of claim 65 wherein said outer material is loosely connected to said inner porous tube.

74. The vascular graft prosthesis of claim 65 wherein said outer material is connected at pre-defined points along a length of said inner porous tube.

75. The vascular graft prosthesis of claim 65 wherein said outer material comprises a combination of stiff and elastic material.

76. The vascular graft prosthesis of claim 65 wherein a portion of said fabric-reinforcing sock is biodegradable.

77. The vascular graft prosthesis of claim 65 wherein a portion of said inner porous tube is biodegradable.

78. The vascular graft prosthesis of claim 65 further comprising an outer layer of material surrounding the adventitial material and inner porous tube.

79. The vascular graft prosthesis of claim 75 wherein said stiff and elastic materials are in contact with each other so that when said elastic material is stretched it takes the initial strain while said stiff material starts to un-bundle, then producing the non-linear elastic response.

80. The vascular graft prosthesis of claim 75 wherein said stiff and elastic materials are combined as a mesh.

81. The vascular graft prosthesis of claim 75 wherein said stiff and elastic materials are combined as a spiral wind.

82. The vascular graft prosthesis of claim 75 wherein said combination of stiff and elastic materials is attached loosely to a surface of said inner tube.

83. The vascular graft prosthesis of claim 75 wherein said combination of stiff and elastic materials is wound around said tube structure.

84. The vascular graft prosthesis of claim 75 wherein said adventitial outer material comprises a geometrical construction of reinforcing textile fabric structure woven together in a tubular form that allows said textile fabric to increase its stiffness with strain.

85. The vascular graft prosthesis of claim 75 wherein said adventitial outer material comprises a geometrical construction of reinforcing textile fabric structure knitted together in a tubular form that allows said textile fabric to increase its stiffness with strain.

86. The vascular graft prosthesis of claim 75 wherein said adventitial outer material comprises a geometrical construction of reinforcing textile fabric structure braided together in a tubular form that allows said textile fabric to increase its stiffness with strain.

87. The vascular graft prosthesis of claim 75 wherein said adventitial material is a textile fabric structure that is woven together as a weave having at least one set of yarns interlaced at angles to each other.

88. The vascular graft prosthesis of claim 87 wherein said textile fabric structure is woven together as a knit having inter-meshed loops of yarn in either weft or warp form.

89. The vascular graft prosthesis of claim 87 wherein said textile fabric structure is woven together as a braid having at least three yarns crossed over each other sequentially.

90. The vascular graft prosthesis of claim 87 wherein said textile fabric structure is a non-woven structure formed by needle-felting yarn into said structure.

91. A computer implemented method of designing a vascular graft prosthesis having desired mechanical characteristics, which mimic the characteristics of natural vessels, comprising the steps of:
entering parameters of fabric graft material and graft data into an encoding processor;
implementing a plurality of computer implemented optimization algorithms which implement a numerical composite graft model analysis and numerical composite circumferential and longitudinal tensile model analyses on a number of parameters; and
forming new data generations using the optimization algorithms performing iterations until desired mechanical characteristics are achieved.

92. A method of manufacturing a vascular graft prosthesis having a bi-layer wall structure configured to optimize mechanical compliance to a host vessel, comprising the steps of:
forming an inner graft structure of a first material; and
attaching an adventitial material to the inner graft structure, wherein the adventitial material is more elastic and less stiff than the first material of the inner graft structure and is characterized by a non-linear elastic response.

93. The method of claim 92 further comprising the step of attaching an outer layer of material to the structure formed by the inner graft structure and the adventitial material.

94. A method of manufacturing a vascular graft prosthesis having a bi-layer wall structure configured to optimize mechanical compliance to a host vessel, comprising the steps of:
performing computer implemented steps of designing a vascular graft prosthesis including entering parameters of fabric material and graft data into an encoding processor, executing a plurality of computer implemented calculations and models on a plurality of parameters, and forming new data generations using the calculations performing iterations until a desired characteristic is met;
using the outcome of the design steps to form an inner material structure which allows uninterrupted cellular growth; and then
surrounding the inner material structure in contacting relation with an outer adventitial material, wherein the outer adventitial material is characterized by a non-linear elastic response to achieve specific compliance values.

95. A method of designing a vascular graft prosthesis having a bi-layer wall structure configured to optimize mechanical compliance to a host vessel, comprising the steps of:
performing computer implemented steps using entering parameters of fabric material and graft data in an encoding processor, executing a plurality of computer implemented calculations and models on a plurality of parameters, and forming new data generations using the calculations performing iterations until a desired compliance value characteristic is met for an outer adventitial portion of a graft prosthesis; and
using the outcome of the computer implemented steps to form an inner material structure of the graft which allows uninterrupted cellular growth and which when formed used inside of the outer adventitial portion provides a graft prosthesis which demonstrates desired characteristics of mechanical compliance for use in a specific host vessel.

96. A method of designing an inner layer of a vascular graft prosthesis having a bi-layer wall structure configured to optimize mechanical compliance to a host vessel, comprising the steps of:
performing computer implemented steps using entering parameters of fabric material and graft data in an encoding processor, executing a plurality of computer implemented calculations and models on a plurality of parameters, and forming new data generations using the calculations performing iterations until a desired compliance value characteristic is met for an outer adventitial portion of a graft prosthesis; and
using the outcome of the computer implemented steps to form an inner material structure of the graft which allows uninterrupted cellular growth and which when formed used inside of the outer adventitial portion provides a graft prosthesis which demonstrates desired characteristics of mechanical compliance for use in a specific host vessel.

97. A method of manufacturing a vascular graft prosthesis having a bi-layer wall structure configured to optimize mechanical compliance to a host vessel, comprising the steps of:
performing computer implemented steps of designing a vascular graft prosthesis including entering parameters of fabric and graft data in an encoding processor, executing a plurality of computer implemented calculations and models on a plurality of parameters, and forming new data generations using the calculations performing iterations until a desired compliance value characteristic is met for an outer adventitial portion of the graft;
using the outcome of the design steps to form an inner material structure which allows uninterrupted cellular growth and;
attaching the outer adventitial portion to the inner material, wherein the adventitial material is characterized by a non-linear elastic response to achieve specific compliance values.

98. A method of manufacturing a vascular graft prosthesis having a bi-layer wall structure configured to optimize mechanical compliance to a host vessel, comprising the steps of:
implementing computer implemented steps of designing a vascular graft prosthesis including entering parameters of fabric and graft data into an encoding processor, implementing a plurality of computer implemented optimization algorithms, which implement graft numerical and mathematical model analyses and numerical and mathematical circumferential and longitudinal tensile model analyses on a number of parameters; and forming new data generations using numerical algorithms performing iterations until desired mechanical compliance characteristics are met;
using the outcome of the design steps to form a tube structure having a circumferential wall with interconnecting pores in the tube wall; wherein porosity is optimized to maximize uninterrupted cellular growth; and then
attaching an outer adventitial material to the tube structure, wherein the adventitial material is characterized by a non-linear anisotropic fabric-reinforcing sock having properties that increase stiffness with strain and being characterized by a non-linear elastic response to achieve specific compliance values.

99. A vascular graft prosthesis having a wall structure configured to optimize mechanical compliance, diameter and wall compression and to prevent over-dilatation of the prosthesis to a host vessel, comprising:
an inner material shaped as a tubular structure which allows cellular growth; and
an outer adventitial material positioned around the inner material, said adventitial material being characterized by a non-linear elastic response having a β value, which is matched to the optimal β value for that portion of the natural host tissue.

100. The vascular graft prosthesis of claim 99 in which the β value is age adjusted for a specific host.

101. A method of manufacturing a vascular graft prosthesis having a bi-layer wall structure configured to optimize mechanical compliance to a host vessel, comprising the steps of:
forming an inner graft structure;
determining the optimal β value for the graft at the location of the host vessel;
manufacturing an outer adventitial material having a characteristic non-linear elastic response which allows the graft prosthesis to have a β value that substantially matches the optimal β value; and
surrounding the inner graft structure with the outer adventitial material.

102. A method of manufacturing a vascular graft prosthesis having a bi-layer wall structure configured to optimize mechanical compliance to a host vessel, comprising the steps of:
determining the optimal β value for the graft at the location of the host vessel;
manufacturing an outer adventitial material having a characteristic non-linear elastic response, which allows the graft prosthesis to have a β value that substantially matches the optimal β value;
forming an inner graft structure; and
surrounding the inner graft structure with the outer adventitial material.

103. A method of designing a vascular graft prosthesis having a bi-layer wall structure configured to optimize mechanical compliance to a host vessel, comprising the steps of:
performing computer implemented steps of designing a vascular graft prosthesis including entering parameters of fabric and graft data in an encoding processor which includes time dependent features replicating tissue ingrowth mechanical impact on the fabric, executing a plurality of computer implemented calculations and models on a plurality of parameters, and forming new data generations using the calculations performing iterations until a desired compliance value characteristic is met for an outer adventitial portion of the graft;
using the outcome of the design steps to form an inner material structure which allows uninterrupted cellular growth and;
attaching the outer adventitial portion to the inner material, wherein the adventitial material is characterized by a non-linear elastic response to achieve specific compliance values.

104. A method of making a vascular graft prosthesis having a multi-layer wall structure configured to optimize mechanical compliance to a host vessel, comprising the steps of:
performing computer implemented steps of designing a vascular graft prosthesis including entering parameters of fabric and graft data in an encoding processor which includes time dependent features replicating tissue ingrowth mechanical impact on the fabric and degradation of the fabric and graft while tissue ingrowth occurs, executing a plurality of computer implemented calculations and models on a plurality of parameters, and forming new data generations using the calculations performing iterations until a desired compliance value characteristic is met for an adventitial portion of the graft;
using the outcome of the design steps to form an inner material structure which allows uninterrupted cellular growth and;
attaching the adventitial portion to the inner material, wherein the adventitial material is characterized by a non-linear elastic response to achieve specific compliance values.
